(19) United States
(12) Patent Application Publication
(10) Pub. No.: US 2003/0064116 A1
(43) Pub. Date: Apr. 3, 2003

(54) Nutritious compositions and food components

(76) Inventors: Albert Crum, Brooklyn Heights, NY (US); D. Dorothea Zivkovic, Cincinnati, OH (US)

Correspondence Address: KLAUBER & JACKSON, 411 HACKENSACK AVENUE, HACKENSACK, NJ, 07601

(21) Appl. No.: 10/085,994

(22) Filed: Sep. 26, 2001

Related U.S. Application Data
(63) Continuation of application No. 09/371,812, filed on Aug. 11, 1999, now abandoned.
Application No. 09/371,812 is a continuation-in-part of application No. 09/095,383, filed on Jun. 10, 1998, now abandoned.
(60) Provisional application No. 60/052,995, filed on May 27, 1997.

(51) Int. Cl. A61K 35/78

(52) U.S. Cl. 424/725

(57) ABSTRACT

This invention describes a nutritional composition containing selenium, colostrum and whey. Composition can be used alone or in combination with food or drink.

### RELATED APPLICATIONS

This application is a continuation in part of copending and commonly owned patent application Ser. No. 09/095,383 filed Jun. 10, 1998 which is a continuation of Provisional Application Serial No. 60/052,995 filed Jun. 10, 1997.

### BACKGROUND OF INVENTION

There are at the present time hundreds, perhaps thousands of food supplement compositions. These compositions are all designed to enhance the nutritional value of the food consumed by mammals including humans and animals. These food supplements often comprise mixtures which if administered orally or parenterally are intended to bring the amount of vitamins, minerals, amino acids and other materials required for proper nutrition in mammals to desired values. They are particularly valuable as sources of essential amino acids which may or may not be in foods but, in any event are destroyed or not synthesized by the metabolic processes of the mammalian body. Eight such amino acids are known. These include for example, leucine, isoleucine and phenylalanine.

The compositions may also contain small amounts of iron, zinc, calcium and other metals, non-metals and other ingredients thought to be necessary for proper nutrition. Some such supplements may contain as many as twenty different ingredients including natural and artificial flavoring agents, natural and artificial colors, binders, fillers and similar materials normally employed in such compositions in the usual amounts.

Glututhione is a tripeptide and a major reducing agent in the mammalian body. Its chemical structure is:

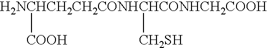

or, more simply

GLU-CYS-GLY

Its chemical name is χ-glutamyl-cysteinyl-glycine.

Like many other small peptides in the mammalian body, it is not synthesized by procedures involving DNA, RNA and ribosome. Rather, it is synthesized from the amino acids available in the body by procedures utilizing enzymes and other body components such as adenosine triphosphate as an energy source.

Glutathione has many functions which aid in the proper functioning of the mammalian body. One of its principal functions is as an antioxidant to reduce hydrogen peroxide to minimize its deleterious effects. Another is to scavenge free radicals. It has several other well substantiated detoxification activities.

The reduction of hydrogen peroxide is shown by the following simplified reaction sequence in which G represents glutathione, SH is the sulfhydryl group of the cysteine moiety and NAHD is an energy source:

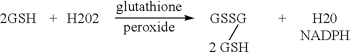

As will be seen, glutathione is regenerated after reducing hydrogen peroxide to water. Failure to reduce hydrogen peroxide has many undesirable consequences as is well known in the art.

Unfortunately, oral administration of GSH is not possible because it is promptly hydrolyzed by the gastric juice and by enzymes in the intestinal system.

It is extremely difficult to introduce glutathione into immune system cells and body tissue. Yet, accomplishing this is vital, because mammalian immune system cells, such as lymphocytes, natural killer cells (NK), and macrophages, depend on glutathione to accomplish their toxin surveillance and detoxification effectively. Glutathione is synthesized in a series of enzymatic steps—the rate limiting step involving χ-glutanyl cysteine synthetase and intracellular cysteine.

Selenium is a trace metal known to be essential for the activity of glutathione. Selenium compounds are one of three essential components in the compositions of this invention.

The selenium compounds employed in accordance with the present invention are water soluble organic or inorganic compounds containing selenium in a form capable of being absorbed by the body. Preferably, the compounds are salts containing selenium in the form of the selenite or selenate anions. In the practice of the present invention, the therapeutic compositions are formulated so that said selenium compound are present to certain specific amounts and concentrations so that the selenium content is not toxic, yet nutritionally useful.

In accordance with the present invention, there are provided nutritional compositions for use in mammals, including humans, comprising an effective but non-toxic amount of a water soluble organic or inorganic selenium compound comprising an effective, non-toxic amount of selenium, suitable up to about 0.00001% of elemental selenium by weight, based on the total weight of the composition in the finished dosage form. Typically, the composition may contain from about 0.000005 to about 0.00001% by weight elemental selenium.

Selenium occurs in a number varying valence forms. For example, selenium compounds in which the selenium has a +4 valance or a +6 valence, usually as the selenite and selenate ions, may be utilized in the compositions of this invention. Among the selenite and selenate forms, the preferred compounds utilized in the compositions of this invention are the water soluble alkali metal salts thereof, and particularly, the sodium and potassium salts, that is, sodium and potassium selenite and selenate. Organic compounds of selenium are preferred for use in the compositions of this invention. For example, selenium compounds of cystine and methionine, as well as the aliphatic mono- and di-selenodicarboxylic acids having about 7 to 11 carbons in the carbon chain may be used. Particularly useful acids of this group include monoseleno-11,11′-di′n-undecanoic acid, diseleno-4,4′-di-n-valeric acid, diseleno-11,11′-di-n-undecanamide and selenium amino acid chelate. It is to be understood, however, that the particular organic forms of selenium compounds set forth herein are not to be considered limitative. Other organic selenium compounds which exhibit the desired activity and are compatible and non-toxic can be used in the practice of this invention.

It is a critical aspect of this invention that the selenium in the form present in the composition be capable of being absorbed by the tissue of the body to be treated. It is noted that water insoluble selenium compounds are not generally absorbed on this level.

### BRIEF DESCRIPTION OF THE INVENTION

The compositions of this invention comprise as essential ingredients ingestible selenium compounds, whey and bovine or caprine colostrum. All of the products are commercially available.

The use of the compositions alone or with other inert or nutritionally useful components increases the immune response of the mammalian body to invasion of infective organisms.

Whey is the curd-free portion of milk that remains after the production of cheese. It is a by-product in the form of an amalgamation of low molecular weight proteins that remains after the casein fraction is removed with the curd. It is a good source of cysteine, glutamine and glycine which are made available for the synthesis of glutathione. Its other components and physiological activities are well known to those skilled in the art. It is commercially available as WPC, “whey protein concentrate” and WPI, “whey protein isolate”. It is also available in a denatured form which can be employed in this invention although it is not preferred. Undenatured protein which in its natural three dimensional structure is much preferred since in its natural configuration it is much better situated to initiate the natural biological reactions which take place in the mammalian body.

Colostrum is a unique combination of beneficial nutrients, including carbohydrate, fat, and amino acids. Colostrum also contains natural vitamins and minerals, which, although not abundant in quantity, are highly bioavailable.

Colostrum, its components and its many beneficial activities are well known to the skilled artisan.

Although the invention is not so limited, the preferred colostrums are bovine and caprine. They are commercially available.

### DETAILED DESCRIPTION OF THE INVENTION

The invention comprises novel nutritious compositions containing whey, colostrum and an ingestible selenium compound such as selenium methionine, selenium amino acid chelate or the others named above. The compositions may be used alone. They may be mixed with a variety of comestibles such as yogurt, ice cream, milk shakes and other dairy products. They may be consumed orally as tablets, capsules and the like. They may be prepared in powder form to spread over foods before ingestion. They may be utilized as components of cooked foods. Many other procedures for utilizing the compositions of this invention will occur to the skilled artisan.

For convenience, the term “selenium” is used in this disclosure and claims to refer to any nontoxic selenium compound employed in the compositions of this invention.

As stated above, glutathione cannot be administered orally since it will be hydrolyzed. One of the surprising discoveries of this invention is that whey and colostrum can be administered orally together with selenium to provide many beneficial effects. The proteinaceous components provide the necessary amino acids to synthesize glutathione once they are absorbed through the intestinal wall. The selenium is readily absorbed and is available to catalyze the reactions by which glutathione functions as an antioxidant or other beneficial purposes.

It is surprising to find that these novel compositions readily achieve their desired result since, despite the vast literature on the isolation, structure and properties of these materials, they have not heretofore been combined to form nutritious compositions or food supplements, nor have they been suggested for such combinations.

Although the products of the invention may be beneficially employed with mammals of any age, they are especially useful for older humans since the production of glutathione decreases with age.

As suggested above, the products of the invention may be provided in any convenient form as with comparable products presently available commercially. Thus they may be employed as powders, capsules, tablets, or in various forms including solutions, suspensions and emulsions. The compositions may additionally contain coloring agents, stabilizing agents, starch or any of a number of other substantially inert components normally employed in the field. If desired, a therapeutic ingredient may also be included in the dosage form. These may include antibiotics, hypertensive agents, hypotensive agents, steroidal antiinflammatory agents or non-steroidal antiinflammatory agents—so called NSAIDS. The guiding criteria is that the additive should not adversely affect the beneficial properties of the principal components.

The compositions may be provided in bulk or unit dosage form. For addition to solid foods or drinks, bulk form will be most convenient.

Wide variations of the amounts of whey and colostrum are possible without adverse effects, the products will be formed with about 5 percent to about 95 percent by weight based on the total weight of the composition of each of colostrum and whey.

Since the products are useful with any mammal, from the smallest to the largest, unit dosage forms may vary from as low as 0.01 grams to 1000 grams. However, 0.5 grams to 20 grams is normally beneficial for must humans.

As stated above, the compositions may optionally contain other ingredients often found in nutritional formulas. These include, for example vitamins and minerals.

The compositions may be employed as an ingredient in foods to enhance their nutritional value. These include liquid foods such as milk shakes or the various liquid dietary aids presently commercially available. it also may be included in solid foods such as breads, cakes, cookies, cupcakes, etc. They are useful as powders to simply shake on foods such as breads and salads.

Several usages of the compositions of the invention are shown in the following examples which are to be considered as illustrations of the invention but not as limitations since many apparent variations thereof are possible without departing from the spirit or scope thereof.

The compositions are prepared using the normal procedures for such products. No special steps are necessary.

### EXAMPLE 1

A composition of the invention is prepared in powder form by mixing:

500 mcg of bovine cholostrum

2 gm. of whey

10 mcg of selenium as selenium amino acid chelate

The mixture is ground into a fine powder which is added as a nutritional supplement to a mixed vegetable salad.

The composition is mixed with a milk shake of any flavor containing about eight ounces of milk to enhance its nutritional value.

The composition is mixed with eight ounces of yogurt to provide a food product of increased nutritional value.

### EXAMPLE 2

A composition useful as an additive to foods is prepared by mixing:

500 mcg of caprine cholostrum

3 gm. of whey protein concentrate

12 mcg of diseleno-11,11′di-n-unedecanamide

The mixture is dried under vacuum and ground into a powder suitable for sprinkling on foods.

About 0.25 grams of this same mixture is added to a capsule suitable for oral ingestion.

What is claimed is:

1. A nutritional composition comprising from about 5% to about 95% whey, from about 5% to 95% colostrum and sufficient selenium compound to provide from about 0.000005% to about 0.00001% selenium, the percent by weight of each component based on the total weight of the composition.

2. The composition of claim 1 in bulk form.

3. The composition of claim 1 in dosage unit form.

4. An edible food containing a nutritionally effective amount of a composition comprising from about 5% to about 95% whey, from about 5% to 95% colostrum and sufficient selenium compound to provide from about 0.000005% to about 0.00001% selenium, the percent by weight of each component based on the total weight of the composition.

5. An edible food of claim 3 wherein the edible food is a milk shake.

6. An edible food of claim 3 wherein the edible food is yogurt.

7. A nutritional composition comprising from about 5% to about 95% whey, from about 5% to 95% colostrum and sufficient selenium compound to provide from about 0.000005% to about 0.00001% selenium, the percent by weight of each component based on the total weight of the composition in the form of a tablet or a capsule.
